(19) United States
(12) Patent Application Publication
(10) Pub. No.: US 2025/0122654 A1
(43) Pub. Date: Apr. 17, 2025

(54) ELASTIC NONWOVEN SHEET

(71) Applicant: FIBERTEX PERSONAL CARE A/S, Aalborg Ost (DK)

(72) Inventor: Morten Rise HANSEN, Aalborg (DK)

(73) Assignee: FIBERTEX PERSONAL CARE A/S, Aalborg Ost (DK)

(21) Appl. No.: 18/688,378

(22) PCT Filed: Oct. 25, 2022
(86) PCT No.: PCT/EP2022/079723

§ 371 Date: Mar. 1, 2024

(30) Foreign Application Priority Data
Nov. 10, 2021 (EP) EP21207486

(51) Int. Cl. D04H 1/559; A61F 13/49; D04H 1/4291; D04H 1/4374; D04H 1/4382; D04H 1/4391; D04H 1/541; D04H 1/56

(52) U.S. Cl. CPC D04H 1/559; A61F 13/4902; D04H 1/4291; D04H 1/4374; D04H 1/43828; D04H 1/43832; D04H 1/43835; D04H 1/43918; D04H 1/5412; D04H 1/5414; D04H 1/5418; D04H 1/56; D10B 2321/022; D10B 2401/061; D10B 2401/063; D10B 2509/026

(57) ABSTRACT

The invention relates to nonwoven sheets that have an ability to elastically stretch in machine direction to a significant extent, while showing low elongation in cross machine direction. The invention also relates to a method for making such sheets.

The invention relates to nonwoven sheets that have an ability to elastically stretch in machine direction to a significant extent, while showing low elongation in cross machine direction. The invention also relates to a method for making such sheets.

Nonwoven sheets are used in the hygiene industry as materials for making baby diapers and adult incontinence products on a large scale. In many instances, for example to make back-ears in open diapers or waist belt portions in diaper pants, elastically stretchable materials are required and standard nonwoven sheets do not meet that requirement.

Traditional approaches for providing elastically stretchable materials based on nonwoven sheets generally involved pleating nonwoven sheets on an elastically stretchable element like a lycra strand. More modern approaches aim to provide nonwoven sheets having an inherent ability to elastically stretch by combining an elastically stretchable nonwoven layer, comprising fibers formed from a thermoplastic elastomer material, with stretchable but not elastic facing layers. The nonwovens disclosed in WO 2020/187540 A1 or EP 3 715 517 A1 can be mentioned as examples.

In typical production methods of adult or baby diaper pants, the circumferential direction of the diaper is usually the machine direction (MD) of the nonwoven sheets used, and the up-down direction of the diaper is usually the cross-machine direction (CD) of the nonwoven sheet used. In these cases, it is advantageous for materials to exhibit a significant elastic stretch in machine direction to allow diaper pants to be elastically stretched in circumferential direction for a good fit, while showing low elongation in cross-machine direction to avoid an excessive stretch of the diaper when pulling it up. This requirement runs counter the fact that in spunbonded nonwoven sheets, which nowadays are industry standard for several different diaper materials and applications, the average fibers orientation is in machine direction, which usually renders elongation lower in machine direction than in cross-machine direction.

There is hence a need in the hygiene industry for nonwoven sheets having a high inherent ability to elastically stretch in machine direction, while having low elongation in cross-machine direction.

In this context, the present invention relates to a method for making a multilayer nonwoven sheet that is elastically stretchable in machine direction, the method comprising: providing an elastically stretchable nonwoven sheet comprising at least two layers of nonwoven materials, wherein one layer is an elastically stretchable nonwoven comprising spunbonded elastic fibers formed from a thermoplastic elastomer polymer material, wherein one layer is a stretchable facing layer comprising spunbonded crimped multicomponent fibers; providing a low elongation nonwoven sheet having a lower elongation at break at least in cross-machine direction when compared to the elastically stretchable nonwoven sheet; and co-feeding the elastically stretchable nonwoven sheet and the low elongation nonwoven sheet to a bonding station where the sheets are joined by melt-bonding in pre-defined patterns, wherein the elastically stretchable nonwoven sheet is fed to the bonding station under a pre-tension in machine direction and hence in a machine-directionally pre-stretched state, while the low CD elongation nonwoven sheet is fed to the bonding station either under no pre-tension, or under a pre-tension that leads to a machine-directional pre-stretch that is less than the machine-directional pre-stretch of the elastically stretchable nonwoven sheet.

The degree of machine-directional pre-stretch of the elastically stretchable nonwoven sheet can be such that, for example, the sheet is pre-stretched by 40-160%, preferably by 60-140%, more preferably by 80-120% of its original dimension.

Preferably, also in terms of absolute force and irrespective of a resulting pre-stretch, the machine-directional pre-tension, if any, of the low elongation nonwoven sheet is lower than the machine-directional pre-tension of the elastically stretchable nonwoven sheet. The degree of pre-tension and pre-stretch, absolutely and relatively, of the sheets co-fed to the bonding station depends on the kind of sheets and the kind of bonding station, with variants being discussed below. In any case, however, the inventive method leads to a configuration where a contraction of the layers of the resulting multilayer sheet, which go back to the elastically stretchable nonwoven sheet, leads to a contraction of the low elongation nonwoven sheet, between the bonding points, that may result in pleating patterns and other repeating structural changes that account for a reservoir allowing for a more or less unopposed machine directional stretch at least to the degree of prior contraction. In cross-machine direction, on the other hand, there is no such reservoir, such that the multilayer sheet as a result is elastically stretchable in machine direction to a significant degree, while showing low elongation in cross-machine direction.

As used herein, the term “low elongation” in characterizing the nonwoven sheet co-fed into the bonding station with the elastically stretchable nonwoven sheet is not meant to specify a certain absolute number of elongation. Generally, this nonwoven sheet shall have an elasticity and elongation at break in cross-machine and preferably also machine direction that will be lower than the one of the elastically stretchable nonwoven sheet. In terms of absolute numbers, elongation at break of this isolated layer as fed could be below 150% and preferably below 100% in both machine and cross-machine direction when measured according to WSP 110.4. The tensile strength in machine direction could be greater 15 N/50 mm and preferably greater 20 N/50 mm, the tensile strength in cross-machine direction greater 5 N/50 mm and preferably greater 10 N/50 mm.

In a preferred embodiment, the elastically stretchable nonwoven sheet and the low elongation nonwoven sheet that are co-fed to the bonding station under different pre-stretch are both pre-bonded, preferably by a pattern of melt-bonding points.

Preferably, the low elongation nonwoven sheet is a spunbonded sheet or at least comprises a layer of spunbonded sheet. More preferably, the low elongation nonwoven sheet is a spunbonded sheet formed from uncrimped monocomponent fibers. A preferred material for forming these fibers is polypropylene, but also polypropylene copolymers (co-PP), like a poly (propylene-ethylene) copolymer, or polyethylene (PE) can be used in embodiments.

The crimped multicomponent fibers of the stretchable facing layer of the elastically stretchable nonwoven sheet can be bicomponent fibers of any asymmetric cross-sectional distribution. Preferred are side-by-side fibers, but they can also be of eccentric-sheath-core or other known configuration.

In one embodiment, the components of the bicomponent fiber are both polypropylene, with the polypropylene differing from each other in properties such that a crimp in the fibers results. Facing layers comprising crimped bicomponent fibers of polypropylene-polypropylene-configuration are used, for example, in the sheets of EP 3 715 517 A1.

In another embodiment, at least one of the components of the crimped multicomponent fibers is a propylene-α-olefin copolymer material (co-PP). This has been found out to potentially lead to an increase in overall elasticity of the sheet, especially in machine direction (MD). The α-olefin co-forming the copolymer with the propylene is preferably ethylene. In other words, the copolymer is preferably a poly (propylene-ethylene) copolymer. Likewise preferably, the copolymer is a random copolymer. The other one of the components of the crimped multicomponent fiber is preferably a polypropylene homopolymer (PP). The co-PP or the homo-PP of the components of such multicomponent fiber may be blended with additional polymers or other additives like slip agents, filler materials, colours or pigments, but should account for more than 50% by weight of the respective component, preferably for more than 75% and more preferably for more than 90%.

Within the crimped bicomponent fibers, the weight ratio of the one component to the other component preferably lies between 20/80 and 80/20, more preferably between 30/70 and 70/30, and yet more preferably between 40/60 and 60/40.

The elastic fibers of the elastically stretchable layer of the elastically stretchable nonwoven sheet can comprise a thermoplastic polyolefin elastomer (TPE-o), preferably a thermoplastic polyolefin elastomer comprising propylene-α-olefin copolymers. Suitable TPE-o materials for use in the context of the present invention are dis-closed in EP 2 342 075 A1. Alternatively or additionally, meaning as a mixture, other thermoplastic elastomer materials like especially thermoplastic polyurethanes (TPU) or styrenic block copolymers (TPE-s) may be used. In one embodiment, up to 20 wt.-% and preferably up to 10 wt.-% of a thermoplastic olefin, such as a homo-polypropylene may be contained in the thermoplastic elastomer material next to the thermoplastic elastomer. In one embodiment, a bicomponent elastic fiber can be formed from two different thermoplastic elastomers, arranged, for example, in a side-by-side or sheath-core configuration.

In one embodiment, the elastically stretchable nonwoven sheet comprises at least one facing layer on either side of the elastic layer, and hence at least three layers overall. This configuration can be advantageous to cover the elastic layer, which tends to be inherently sticky and difficult to handle in production when exposed, on both sides. The additional facing layer may be configured as described above for the first facing layer. The facing layers on the different sides of the elastic layer may be the same, but may also be different. For example, one of the nonwoven facing layers may be a spunbonded nonwoven and the other nonwoven facing layer may be a different spunbonded nonwoven or a meltblown nonwoven.

In another embodiment, the elastic layer of the elastically stretchable nonwoven sheet is exposed on the side that faces the low elongation nonwoven sheet, and after joining the sheets in the bonding stations lies directly adjacent the low elongation nonwoven sheet. This has the advantage of a lesser number of overall layers.

The layers as defined above can comprise the elastic, monocomponent or bicomponent fibers as defined, respectively, in addition to other fibers, but preferably consist of the respective fibers as defined.

The pre-stretch of the elastically stretchable nonwoven sheet may, for example, be adjusted by nip-rolls arranged ahead of the bonding station, and possibly nip-rolls arranged following the bonding station. The nip-rolls can adjust the translation speed of the sheet.

The bonding station comprises at least one calender roll having embossing projections on the surface. Embodiments comprise ultrasonic bonding, where ultrasonic vibrations are introduced to the embossing projections. Other embodiments use thermal bonding where the embossing projections are heated. In embodiments, the sheets are fed to the gap between two counter-rotating rolls, at least one of which is an calender roll.

A specific embodiment of a bonding station comprises a pair of interacting rolls whose surfaces comprise interlocking cross-directional ribs and grooves, and embossing projections arranged along the crests of the ribs and/or grooves on at least one of the rolls. The interlocking ribs and grooves effect a further localized machine-directional pre-stretch of the layers. The bonding together of the sheets while in this state increases the effect that has been described above with respect to the pre-tension difference.

Against the background initially described, the invention further relates to a multilayer nonwoven sheet that is elastically stretchable in machine direction, comprising at least three layers of nonwoven materials; wherein a first layer comprises spunbonded elastic fibers formed from a thermoplastic elastomer polymer material; wherein a second layer comprises spunbonded crimped multicomponent fibers; wherein the layers of the sheet are melt-bonded together by a pattern of bonding points; and wherein the third layer is machine-directionally contracted between the bonding points and shows a pleating pattern and/or other repeating structural changes in machine direction.

The fabric can be obtained by a method of the invention. Preferred aspects of the layers can be taken from the description of the inventive method above.

Preferably, the multilayer sheet has an elongation at break value of higher 100% and preferably of higher than 150% in machine direction when measured according to WSP 110.4. The elongation at break value of the multilayer nonwoven sheet in cross-machine direction, when measured according to WSP 110.4, is preferably lower 100%.

In one embodiment, the elongation at break value of the multilayer nonwoven sheet in machine direction can be higher than the elongation at break in cross-machine direction, when measured according to WSP 110.4.

The tensile strength in cross-machine direction for the multilayer sheet, when measured according to WSP 110.4 shall preferably lie beyond 10 N/50 mm and preferably beyond 15 N/50 mm. The CD tensile strength (at break) of the third layer is significantly governed by the CD tensile strength of the third layer, which, when considered by itself, preferably has a higher tensile strength in cross-machine direction when compared to the first or second layer.

The basis weight of the second layer, and more generally of each crimped fiber-formed facing layer(s) of the fabric (within the elastically stretchable nonwoven sheet of the multilayer sheet), may be between 5-40 g/m², preferably between 8-30 g/m², more preferably between 10-25 g/m² and yet more preferably between 15-20 g/m².

If more than one crimped fiber-formed facing layer is present in the fabric, these can have the same basis weights, or one can have a lower basis weight than the other. The embodiment with different basis weights can be preferred under the rationale that one of the facing layers in a three-layered elastically stretchable nonwoven sheet may, before the stretchable sheet is laminated to the low elongation nonwoven sheet, only have a function except shielding the sticky elastic layer.

The basis weight of the first (elastic) layer may be between 10-150 g/m², preferably between 20-120 g/m² and more preferably between 25 and 100 g/m².

The basis weight of the third (low elongation nonwoven) layer may be between 5-50 g/m², preferably between 8-30 g/m² and more preferably between 10 and 25 g/m².

The nonwoven sheets according to the invention are particularly suited for use in the manufacture of hygiene articles. For example, the nonwoven sheets can be used for the manufacture of a diaper pant comprising the sheet as an elastic waist material. Typical production processes currently employed in the industry in that application would require the material to be able to elastically stretch in MD.

Further details and advantages of the invention will become apparent from the figures and examples described in the following. The figures show:

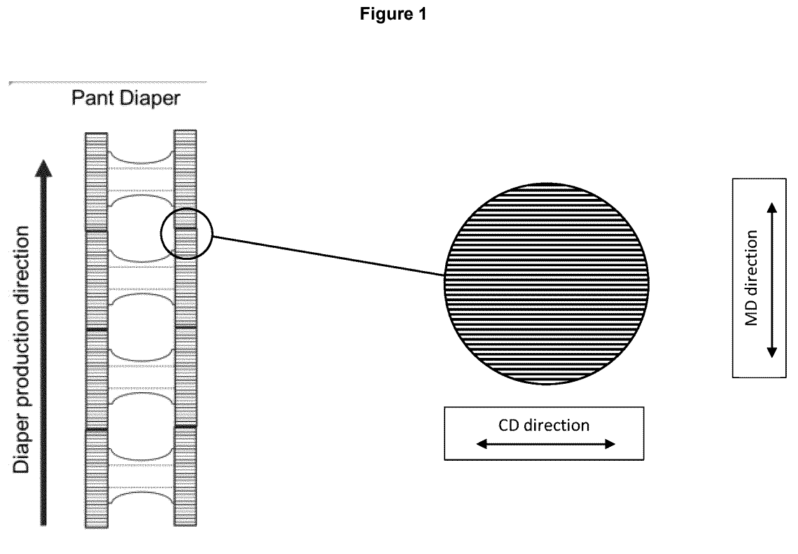

FIG. 1: a production layout of how a nonwoven sheet is usually used in industrial production of pant diapers;

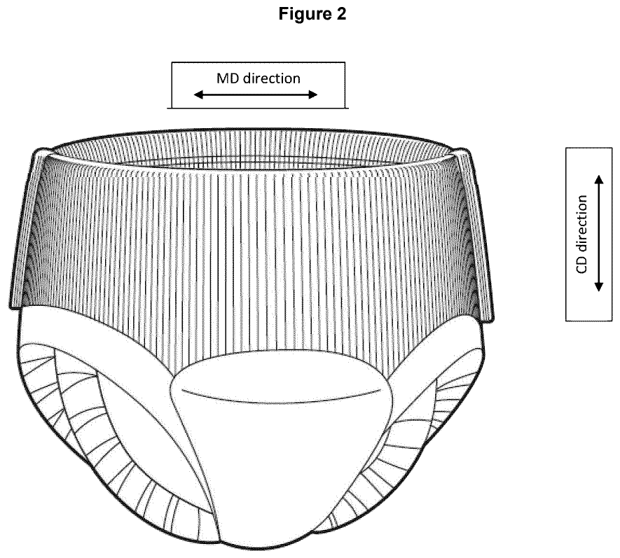

FIG. 2: a pant diaper with cross-machine and machine directions of the nonwoven belt material displayed;

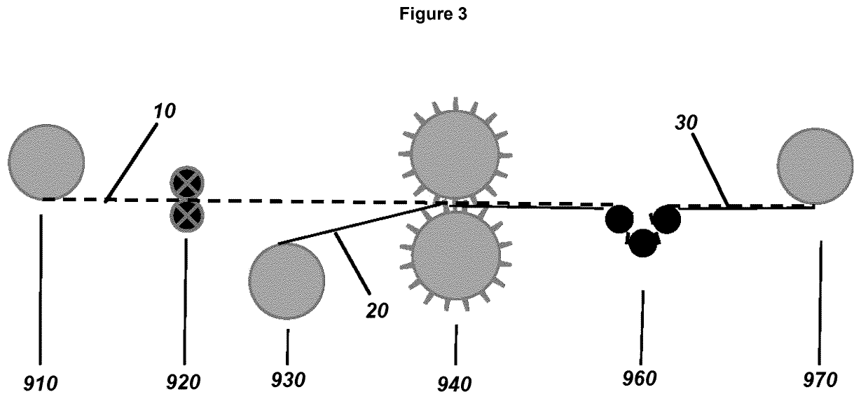

FIG. 3: a machine setup for carrying out the method of the invention in a first example;

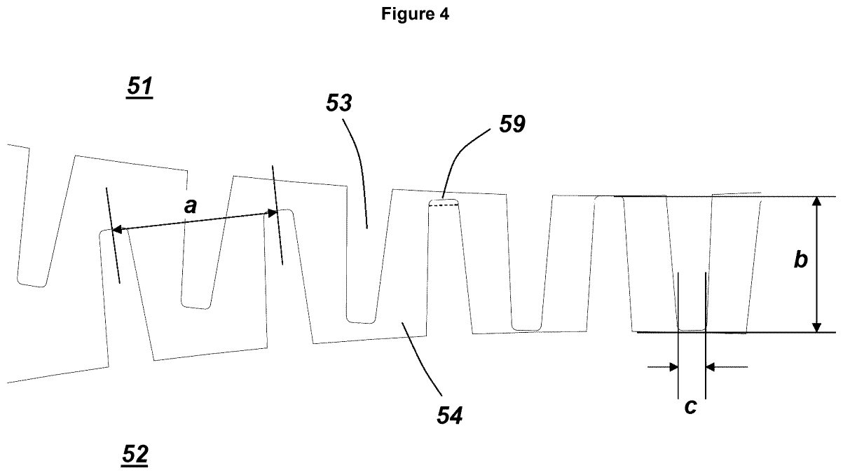

FIG. 4: a schematic illustration of an activation unit for additionally pre-stretching and concurrently bonding the co-fed laminates;

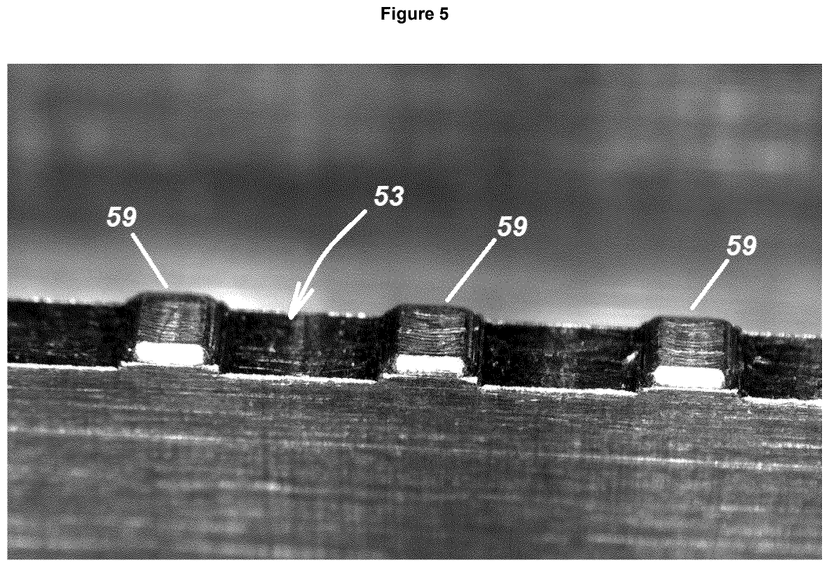

FIG. 5: a picture of a crest line of a rib of a roll as schematically illustrated in FIG. 4;

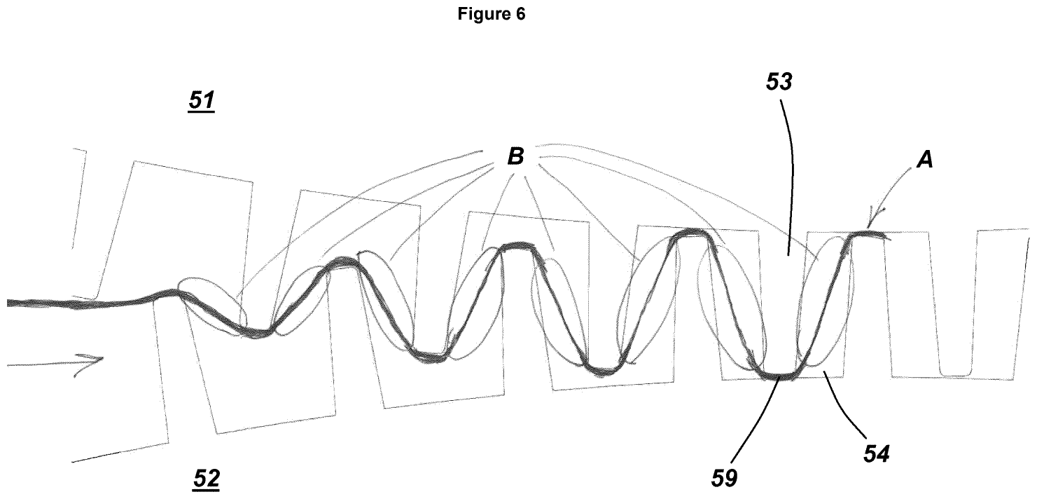

FIG. 6: a schematic illustration of an activation unit as of FIG. 4 in operation;

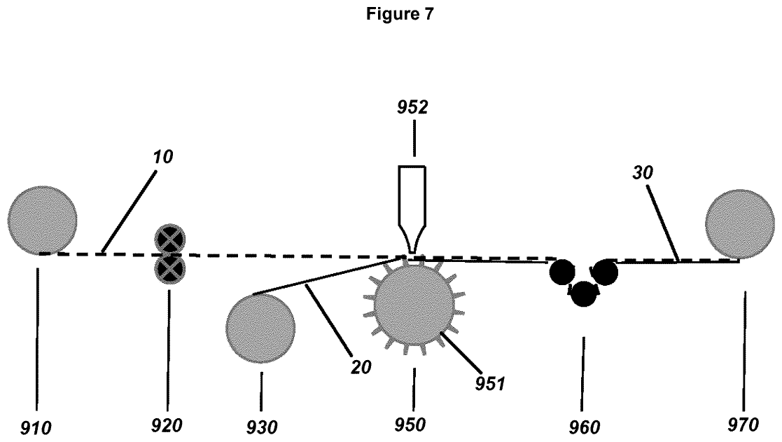

FIG. 7: a machine setup for carrying out the method of the invention in a second example;

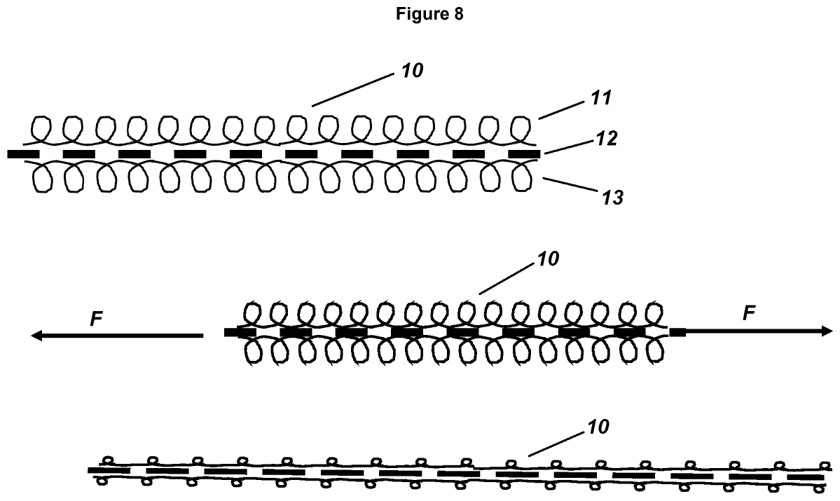

FIG. 8: an illustration of the structural changes in a three-layered elastically stretchable nonwoven sheet when a machine-directional pre-tension is applied;

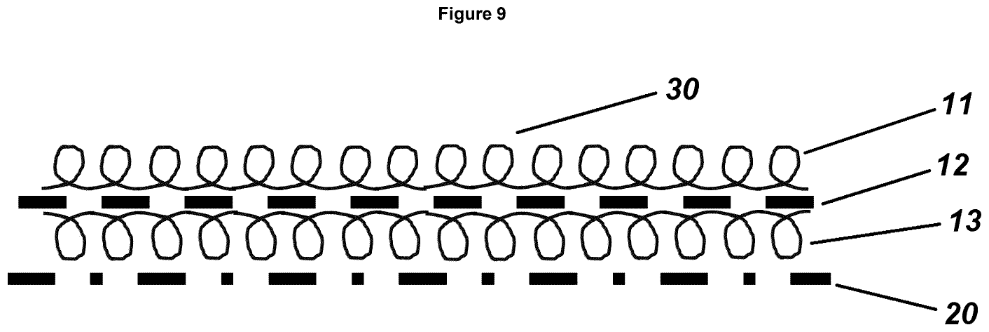

FIG. 9: an illustration of an embodiment of a multilayer nonwoven sheet based on a three-layered elastically stretchable nonwoven sheet;

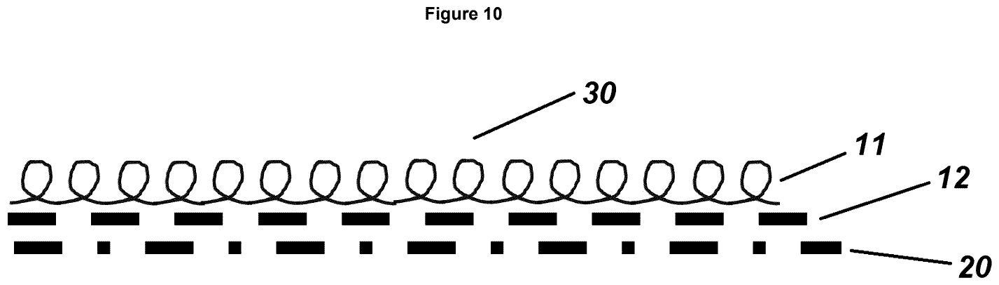

FIG. 10: an illustration of an embodiment of a multilayer nonwoven sheet based on a two-layered elastically stretchable nonwoven sheet; and

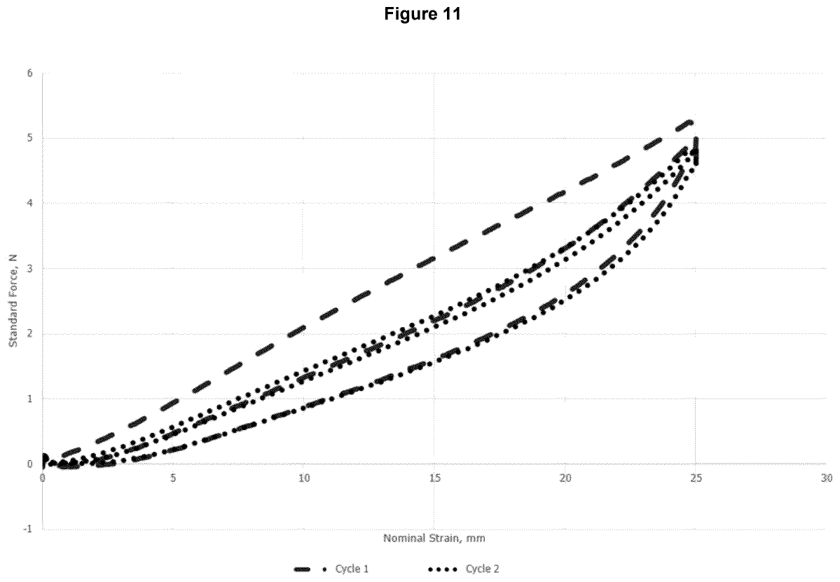

FIG. 11: a hysteresis curve in a machine-directional tensile (stress-strain) diagram that has been recorded for the laminate material of Example 1.

FIGS. 1 and 2 illustrate why it is desirable in pant diaper production to have sheets with elastic properties in machine direction and a relatively low elongation in cross-machine direction.

Specifically, FIG. 1 illustrates a production layout of how a nonwoven sheet is usually used in industrial production of pant diapers. It can be seen that the diaper belts of the diapers derived from the materials are oriented in machine direction.

As diaper pants should at the same time be comfortable to wear, and hence stretchable in the belt portion, and easy to mount, and hence dimensionally stable in up-down-direction, nonwovens optimized to that needs must feature good elastic performance in machine direction, whereas the cross-machine directional elasticity and elongation will have to be relatively low and the cross-machine directional elongation will have to be adequately low in order to survive the force needed to mount the diaper when puling it up to put in the right place.

A pant diaper with the corresponding cross-machine and machine directions of the nonwoven belt material displayed is shown in FIG. 2.

FIG. 3 illustrates and exemplary machine setup for carrying out the method of the invention in a first example, where an elastically stretchable nonwoven sheet is laminated to a regular spunbond nonwoven sheet (as the low elongation nonwoven sheet), the lamination process being carried out without the addition of any adhesive or glue.

In this process, an elastically stretchable nonwoven sheet 10 is being unwound at station 910 into the process with a speed controlled by a pair of nip rolls at 930. A regular (low elongation) spunbond nonwoven sheet 20 is unwound at station 920 and co-fed to a combined activation and bonding process at bonding station 940, described in more detail below with reference to FIGS. 4 to 6. Leaving the station 940, the material is relaxed and processed through a set of so-called banana or spreader rolls at station 960 to control the web path and make sure the material is wrinkle free. After this the resultant multilayer sheet 30 is wound up in finished laminate rolls at station 970.

FIGS. 4 to 6 illustrate an embodiment of a bonding station 940 as used in the example of FIG. 3, which is configured as an activation unit for additionally pre-stretching and concurrently bonding the co-fed sheets. The unit comprises two counter-rotating activation rolls 51, 52 which are configured for additionally stretching the co-fed sheets in machine direction and bonding them in a stretched state.

The picture of FIG. 4 is an enlarged cross-section along a radial plane perpendicular to the roll axis. As apparent from this figure, both rolls 51 and 52 comprise a plurality of regularly spaced ribs 53 on their acting surfaces, between which grooves 54 are formed. The ribs 53 in this embodiment are oriented in cross-machine direction and extend axially over the surfaces of the rolls 51 and 52.

The width “a” of the ribs 53, the depth of engagement “b” and the distance between adjacent ribs 53 “c” controls the extent of the machine directional pre-stretch during activation.

At the crest lines of each rib 53 on both rolls 51, 52 there is a series of embossing projections 59 for bonding together the co-fed sheets while they are stretched, or in other words for bonding together the co-fed sheets simultaneously with an additional stretch activation.

FIG. 5 shows a picture of a crest line of a rib 53 of an activation roll 51, 52 as schematically illustrated in FIG. 4, the rib 53 having embossing projections 59 on its crest line.

FIG. 6 shows a unit as shown in FIG. 4 in operation. From left to right in FIG. 6, the co-fed sheets (shown as one sheet only for illustrative purposes) enters the combined activation and bonding process. As the sheets enter the nip of the two rolls 51, 52 the activation process is initiated with the meshing ribs 53. Due to the progressing engagement of the ribs 53 in the grooves 54 the localized stretch steadily increases up to the center position where the ribs 53 and grooves 54 are fully engaged.

At the maximum engagement point in the center position of the two rolls 51, 52, the embossing projections 59 on a rib 53 of one roll (here: the roll 51) will be in contact with the bottom of the opposite groove 54 of the opposite roll (here: the roll 52) and form a series of bonding points along the crest line of the rib 53, i.e. along a stripe-shaped high-density zone A of the co-fed sheets.

As the material again exits the station 940, the elastic fibers in the elastic layer of the elastically stretchable nonwoven sheet 10 contract and revert the stretch of the material. The sections of the regular (low elongation) spunbond nonwoven sheet 20 that are not attached to the elastically stretchable nonwoven sheet 10 in a bonding point, during this relaxation process, pleat, wrinkle, or get compacted by fiber cramping inside the sheet, and as such maintain the ability to again stretch in machine direction to the extent they have been pre-stretched in the station 940, while maintaining their original low elongation in cross-machine direction.

Again going back to the description of FIG. 3, at station 930, the speed of the elastically stretchable nonwoven sheet 10 before entering the bonding station 940 is controlled and adjusted to a speed lower level than the speed of the activation rolls of station 940, and thereby the speed of the regular (low elongation) nonwoven sheet 20 before entering the bonding station 940, which is on par with the speed of the rolls in station 940. By running the elastically stretchable nonwoven sheet 10 at a lower speed it will enter station 940 at a pre-stretched state and, owing to the configuration of the rolls in station 940, will therein be stretched and activated even more, up too and above 200% elongation.

The activation rolls of station 940 are heated to a degree suitable to form bonds between the elastically stretchable nonwoven sheet 10 and the regular (low elongation) nonwoven sheet 20. A typical temperature range is between 50° C. and 145° C., preferably between 60° C. and 70° C. The temperature between the bottom and top roll can differ from each other.

FIG. 7 a machine setup for carrying out the method of the invention in a second example. The setup is very similar to the setup of FIG. 3, with the only difference that the bonding station 950 is differently configured and only comprises one embossing roll 951, whose surface comprises embossing pins, and an ultrasonic weld tool, more specifically a sonotrode 952, arranged at a small distance above it. The co-fed sheets are lead over roll 951, preferably by a slight bend, and pass through the gap between the roll 951 and the sontrode 952 while lying on the roll 951 surface.

The ultrasonic welding in principle is well-known. The thermoplastic fibers in the sheets are activated by mechanical vibrations created by the sonotrode 952, leading to melting and bonding in pre-defined patterns corresponding to the bonding pins on the roll 951 surface, which focus the energy and hence precisely define the weld spots.

In the machine setup of FIG. 7, when compared to the machine setup of FIG. 3, the pre-stretch is a bit less aggressive.

The nip pressure of the roll(s) at station 940 or 950, in either example described above, can be from 5-100 N/mm, and the embossing projections can occupy a bonding area of 8-20%. The embossing projections can have an area of 0.2 mm² to 2 mm² and can have a rectangular, round, oval or other shape.

FIG. 8 is an illustration that explains the structural changes in a three-layered elastically stretchable nonwoven sheet 10 when a machine-directional pre-tension is applied. The sheet comprises two facing layers 11 and 13 on either side of a sandwiched elastic layer 12. The facing layers 11 and 13 can each be spunbonded fabrics formed from crimped fibers, which render the fabric flexible and more stretchable when compared to a standard spunbonded nonwoven formed from uncrimped fibers. The elastic layer 12 is a spunbonded nonwoven made from elastic fibers formed from a thermoplastic elastomer polymer material.

The uppermost picture of FIG. 8 shows the sheet 10 in an unstretched state. If a longitudinal force F is applied (middle picture of FIG. 8), for example by a lessening of the speed with which the fabric is fed to the bonding station 950 of the machine setup of FIG. 7, the fabric is stretched in machine direction (lower illustration of FIG. 8). If the force goes away, the fabric, by action of the elastic layer 12 automatically retracts again to its original length (uppermost picture of FIG. 8).

If, in the stretched state as shown in the lowest picture of FIG. 8, an additional layer, in form of the regular (low elongation) nonwoven layer 20 is attached to the sheet 10, and if the combined multilayer sheet 30 is then allowed to relax and retract, the layer that goes back to sheet 10 is compacted in machine direction between the bonding points, which may result in pleating patterns and other repeating structural changes that account for a reservoir allowing for a more or less unopposed machine directional stretch at least to the degree of prior contraction. In cross-machine direction, on the other hand, where there is no such reservoir, the layer that goes back to sheet 10 maintains its original (low) elongation.

FIGS. 9 and 10 show multilayer sheets 30 in a final relaxed state, where FIG. 9 illustrates an embodiment of a multilayer nonwoven sheet 30 based on a three-layered elastically stretchable nonwoven sheet 20 comprising two facing layers 11 and 13 and an elastic layer 12, and FIG. 10 an embodiment of a multilayer nonwoven sheet 30 based on a two-layered elastically stretchable nonwoven sheet comprising only one facing layer 11, with the elastic layer 12 being directly adjacent the contracted regular (low elongation) layer 10.

### EXAMPLE 1

The following example describes the making of a four-layered fabric in agreement with the invention in an apparatus as schematically illustrated in FIG. 3.

A three-layered elastically stretchable nonwoven sheet 10, that is configured as schematically shown in FIG. 8 and comprises an elastic spunbonded layer 11 sandwiched between two spunbonded facing layers 12 and 13 formed from crimped bicomponent fibers, is co-fed to a combined activation and bonding unit 940 with a layer of a regular low elongation spunbonded fabric 20 formed from uncrimped monocomponent fibers.

During feed, the elastically stretchable nonwoven sheet 10 was pre-stretched by 100% of its original length by having an ingoing speed to the nip of the rollers of unit 940 of 22 m/min, while the rollers are operated at 44 m/min. Regular low elongation spunbonded fabric 20 is fed at 44 m/min. The depth of engagement “b” in unit 940 was 5 mm (at a total height of the ribs of 5 mm, such that the bonding points on the crst lines engage with the surface of the other roller).

The setup of the sheet 10 was as described in the following Table 1:

TABLE 1

| | | | | | Basis |
|---|---|---|---|---|---|
| | | | | | weight |
| | Type | Polymer 1 | Polymer 2 | Ratio | (gsm) |
| Facing layer 12 | PP/CoPP | 511A | QR674K | 50/50 | 20 |
| Elastic layer 10 | Th-El. | Vistamax 7050BF | | | 40 |
| Facing layer 13 | PP/CoPP | 511A | QR674K | 50/50 | 20 |

The 511A polymer is a polypropylene homopolymer from the company Sabic with a narrow polymer weight distribution (M_{w}/Mₙ is 3,8), a MFR of 25 g/10 min and a Tₘ of 161° C.

The QR674K polymer is an ethylene-propylene random copolymer from the company Sabic with an MFR of 40 g/10 min, a broad molecular weight distribution (M_{w}/Mₙ is 8,5) and a Tₘ of 150° C. It also further contains a clarifier and a slip agent.

The elastic layer was made from a single commercially available TPE-o material Vistamaxx™ 7050FL from ExxonMobil, which is a propylene-based thermoplastic elastomer copolymer with an ethylene content of 13 wt.-% and a melt flow rate of 45 g/10 min.

The bonding pattern of the sheet 10 was an open dot bonding pattern of 12% bonding area with 24 bond sites per cm².

Table 2 below shows properties that have been measured for this sheet 10.

TABLE 2

| | Basis | | MD tensile |
|---|---|---|---|
| | weight | Thickness* | at break** |
| | (gsm) | (mm) | (N/50 mm) |
| Sheet 10 | 77.8 | 0.78 | 30.5 |
| | MD | CD | CD |
| | elongation | tensile | elongation |
| | at break** | at break** | at break** |
| | (%) | (N/50 mm) | (%) |
| Sheet 10 | 249 | 20.8 | 321 |

*Determined according to WSP120.6
**Determined according to WSP 100.4

Sheet 20 was a standard commercially available spunbond nonwoven material made from monocomponent PP fibers. The trade name is A10150KW from the company Fibertex Personal Care A/S. Technical data are as summarized in Table 3 below:

TABLE 3

| | Basis | | MD tensile |
|---|---|---|---|
| | weight | Thickness* | at break** |
| | (gsm) | (mm) | (N/50 mm) |
| Sheet 20 | 15.0 | 0.17 | 28.0 |
| | MD | CD | CD |
| | elongation | tensile | elongation |
| | at break** | at break** | at break** |
| | (%) | (N/50 mm) | (%) |
| Sheet 20 | 70 | 13.0 | 90 |

*Determined according to WSP120.6
**Determined according to WSP 100.4

The resultant CD tensile and elongation at break after laminating the two sheets 10 and 20 together cannot be smaller (tensile) or higher (elongation) than the respective values of the isolated sheet 20. This (wanted) limitation for the cross-machine direction, however, is of no detriment to the laminate's ability to perform well in terms of MD elongation and elasticity. This was experimentally confirmed. Specifically, the data that have been obtained for the resultant sheet 30 are summarized in the following Table 4.

TABLE 4

| | Basis | | MD tensile |
|---|---|---|---|
| | weight | Thickness* | at break** |
| | (gsm) | (mm) | (N/50 mm) |
| Sheet 30 | 105 | 1.43 | 39.2 |
| | MD | CD | CD |
| | elongation | tensile | elongation |
| | at break** | at break** | at break** |
| | (%) | (N/50 mm) | (%) |
| Sheet 30 | 147 | 13.5 | 75 |

*Determined according to WSP120.6
**Determined according to WSP 100.4

FIG. 11 shows a hysteresis curve in a machine-directional tensile (stress-strain) diagram that has been recorded for this laminate material, sheet 30, in agreement with the standard test method ASTM D5459. This curve allows to confirm two parameters of the machine-directional elasticity behaviour.

A first parameter is the permanent deformation, defined as the increase in length, expressed as a % of the original length, by which an elastic material fails to return to the original length after subjected to the extensions prescribed in the test procedure in ASTM D5459. The lower the % of permanent deformation, the better is the elasticity property of the elastic material.

A second parameter is the area between the increasing and decreasing stress-strain curves of a hysteresis plot in a second cycle of an ASTM D5459 test, as expressed in the relative size of the area between the curves (A) in relation to the overall area under the initial increasing curve (A+B), expressed in % [A/(A+B)×100]. It is to calculate the % of the energy dissipated due to internal friction. When the plots during loading and unloading do not coincide, as usually observed in real life materials, this means that a certain amount of energy is lost. The lower the %, the better the elastic property of the material.

For the material tested, from the hysteresis shown in FIG. 11, the permanent deformation after the first cycle has been determined to be below 5% and the area between the increasing and decreasing curves of the second cycle has been measured to be 26,7%. These are very favourable values.

1. A method for making a multilayer nonwoven sheet that is elastically stretchable in machine direction, the method comprising:
providing an elastically stretchable nonwoven sheet comprising at least two layers of nonwoven materials, wherein one layer is an elastically stretchable nonwoven comprising spunbonded elastic fibers formed from a thermoplastic elastomer polymer material, wherein one layer is a stretchable facing layer comprising spunbonded crimped multicomponent fibers;
providing a low elongation nonwoven sheet having a lower elongation at break at least in cross-machine direction when compared to the elastically stretchable nonwoven sheet; and
co-feeding the elastically stretchable nonwoven sheet and the low elongation nonwoven sheet to a bonding station where the sheets are joined by melt-bonding in pre-defined patterns, wherein the elastically stretchable nonwoven sheet is fed to the bonding station under a pre-tension in machine direction and hence in a machine-directionally pre-stretched state, while the low elongation nonwoven sheet is fed to the bonding station either under no pre-tension, or under a pre-tension that leads to a machine-directional pre-stretch that is less than the machine-directional pre-stretch of the elastically stretchable nonwoven sheet.

2. The method of claim 1, wherein the degree of machine-directional pre-stretch of the elastically stretchable nonwoven sheet is such that the sheet is pre-stretched by 40-160% of its original dimension.

3. The method of claim 1, wherein the elastically stretchable nonwoven sheet and the low elongation nonwoven sheet that are co-fed to the bonding station are both pre-bonded.

4. The method of claim 1, wherein the low elongation nonwoven sheet is a spunbonded sheet.

5. The method of claim 1, wherein the components of the bicomponent fiber are all polypropylene.

6. The method of claim 1, wherein one of the components of the crimped multicomponent fibers is a propylene-α-olefin copolymer material (co-PP), and at least another component of the crimped multicomponent fibers is a polypropylene homopolymer (PP).

7. The method of claim 1, wherein the elastic fibers of the elastically stretchable layer comprise a thermoplastic polyolefin elastomer (TPE-o).

8. The method of claim 1, wherein the elastically stretchable nonwoven sheet comprises at least one facing layer on either side of the elastic layer.

9. The method of claim 1, wherein the elastic layer of the elastically stretchable nonwoven sheet is exposed on the side that faces the low elongation nonwoven sheet, and after joining the sheets in the bonding stations lies directly adjacent the low elongation nonwoven sheet.

10. The method of claim 1, wherein the pre-stretch of the elastically stretchable nonwoven sheet is adjusted by using nip-rolls to adjust its translation speed.

11. The method of claim 1, wherein the bonding station comprises at least one calender roll having embossing projections on the surface, which are heated or ultrasonically vibrated.

12. The method of claim 1, wherein the bonding station comprises a pair of interacting rolls whose surfaces comprise interlocking cross-directional ribs and grooves, and embossing projections arranged along the crests of the ribs and/or grooves on at least one of the rolls.

13. A multilayer nonwoven sheet that is elastically stretchable in machine direction, comprising at least three layers of nonwoven materials;
wherein a first layer comprises spunbonded elastic fibers formed from a thermoplastic elastomer polymer material;
wherein a second layer comprises spunbonded crimped multicomponent fibers;
wherein the layers of the sheet are melt-bonded together by a pattern of bonding points; and
wherein the third layer is machine-directionally contracted between the bonding points and shows a pleating pattern and/or other repeating structural changes in machine direction.

14. The multilayer nonwoven sheet of claim 13, wherein the elongation at break value of the multilayer nonwoven sheet in machine direction, when measured according to WSP 110.4, is higher than 100%; and/or wherein the elongation at break value of the multilayer nonwoven sheet in cross-machine direction, when measured according to WSP 110.4, is lower than 100%; and/or wherein the elongation at break value of the multilayer nonwoven sheet in machine direction is higher than its elongation at break in cross-machine direction, when likewise measured according to WSP 110.4.

15. A method of making a hygiene article, said method comprising utilizing the multilayer nonwoven sheet according to claim 13.

16. The method of claim 1, wherein the degree of machine-directional pre-stretch of the elastically stretchable nonwoven sheet is such that the sheet is pre-stretched by 60-140% of its original dimension.

17. The method of claim 1, wherein the degree of machine-directional pre-stretch of the elastically stretchable nonwoven sheet is such that the sheet is pre-stretched by 80-120% of its original dimension.

18. The method of claim 1, wherein the elastically stretchable nonwoven sheet and the low elongation nonwoven sheet that are co-fed to the bonding station are both pre-bonded by a pattern of melt-bonding points.

19. The method of claim 1, wherein the low elongation nonwoven sheet is a spunbonded sheet formed from uncrimped monocomponent fibers.

20. The method of claim 1, wherein one of the components of the crimped multicomponent fibers is a poly (propylene-ethylene) copolymer, and at least another component of the crimped multicomponent fibers is a polypropylene homopolymer (PP).
